(19) United States
(12) Patent Application Publication
(10) Pub. No.: US 2024/0301346 A1
(43) Pub. Date: Sep. 12, 2024

(54) CELLULAR MICROCOMPARTMENTS COMPRISING CELLS OF WHICH THE GENOMIC INTEGRITY IS MAINTAINED AFTER AMPLIFICATION AND PREPARATION METHOD

(71) Applicant: TREEFROG THERAPEUTICS, PESSAC (FR)

(72) Inventors: Maxime FEYEUX, TALENCE (FR); Philippe Joseph COHEN, BLANQUEFORT (FR)

(21) Appl. No.: 18/270,924

(22) PCT Filed: May 11, 2022
(86) PCT No.: PCT/EP2022/062792

§ 371 Date: Aug. 24, 2023

(30) Foreign Application Priority Data
May 11, 2021 (FR) FR2104988

(51) Int. Cl. C12N 5/00; C12N 5/074

(52) U.S. Cl. CPC C12N 5/0012; C12N 5/0696; C12N 2501/727; C12N 2510/00; C12N 2513/00; C12N 2533/74

(57) ABSTRACT

The invention relates to a three-dimensional cellular microcompartment or a three-dimensional cellular microcompartment assembly comprising at least one external hydrogel layer and inside said external layer at least one layer of cells and/or at least one cellular base layer, of which less than 20% of the total population of cells present in the microcompartment or in the microcompartment assembly are cells having at least one mutation.

The invention also relates to a method for producing such a microcompartment or microcompartment assembly.

### TECHNICAL FIELD

The invention relates to maintaining the genomic integrity of cells during their ex vivo division over several cell division cycles, in particular in the context of a three-dimensional cell culture.

### PRIOR ART

Ex vivo cell culture is a field which is generating increasing interest. The cultured cells may be of any type. It may involve both differentiated cells with different phenotypes, progenitor cells and stem cells. A significant advance in cell culture techniques is the introduction of three-dimensional culture systems. Three-dimensional cultures are indeed closer to in vivo natural systems, and can be used for numerous applications in particular in the development of therapies.

However, cell therapy and tissue engineering are conditioned by the availability of industrial quantities of cells which requires having recourse to a large multiplication in the number of cells and therefore a high number of divisions over a short time. In most current cell culture systems, this multiplication leads to the appearance and selection of mutations, in particular harmful, genomic and/or epigenetic functional mutations at each division over numerous cells during the expansion of the culture, thus compromising their use in particular in therapy.

The mutations can be point mutations of the genetic sequence (coding or non-coding, silent or not in terms of peptide sequence), structural variants, epigenetic modifications, or even mitochondrial DNA modifications. Only the mutant cells bearing one or more functional or potentially functional mutations are problematic for the use of the cells in therapy, that is any transmissible genetic or epigenetic modification which confers a gain or loss of function or potential loss of function to cultured cells. It may in particular be an advantage of growth, a decrease in susceptibility to cell death, a modification of the genes involved in tumorigenesis or suppression of tumorigenesis. The most influential mutations are those allowing a clonal expansion of the cells which become dominant in culture.

Examples of particularly recurrent genetic mutations are described in particular in Y. Avior, K. Eggan, N. Benvenisty, Cancer-Related Mutations Identified in Primed and Naive Human Pluripotent Stem Cells. Cell Stem Cell. 25, 456-461 (2019). Among the best known, mention may in particular be made of mutations of the P53 gene (F. T. Merkle, S. Ghosh, N. Kamitaki, J. Mitchell, Y. Avior, C. Mello, S. Kashin, S. Mekhoubad, D. Ilic, M. Charlton, G. Saphier, R. E. Handsaker, G. Genovese, S. Bar, N. Benvenisty, S. A. McCarroll, K. Eggan, Human pluripotent stem cells recurrently acquire and expand dominant negative P53 mutations. Nature. 545, 229-233 (2017)), and mutations by amplification of the 20q11 chromosome region (N. Lefort, M. Feyeux, C. Bas, O. Féraud, A. Bennaceur-Griscelli, G. Tachdjian, M. Peschanski, A. L. Perner, Human embryonic stem cells reveal recurrent genomic instability at 20q11.21. Nature Biotechnology. 26, 1364-1366 (2008)).

The problem of genetic stability and integrity of cultured cells is known and in particular has been widely studied for pluripotent stem cells, such as for example: S. Attwood, M. Edel, IPS-Cell Technology and the Problem of Genetic Instability—Can It Ever Be Safe for Clinical Use? Journal of Clinical Medicine. 8, 288 (2019); or also P. Andrews, Human pluripotent stem cells: genetic instability; or stability: Regenerative medicine, vol. 16, No 2, Mar. 2, 2021. It is also known that mutagenesis is a very present problem for culturing stem cells as soon as they are reprogrammed as described in Ji, S. Ng, V. Sharma, D. Necut, S. Human, M. Sam, Q. Trinh, G. M. Church, J. D. McPherson, A. Nagy, N. N. Batada, Elevated coding mutation rate during the reprogramming of human somatic cells into induced pluripotent stem cells. Stem Cells. 30, 435-440 (2012), and in V. Turinetto, L. Orlando, C. Giachino, Induced pluripotent stem cells: Advances in the quest for genetic stability during reprogramming process. International Journal of Molecular Sciences. 18 (2017), doi:10.3390/ijms18091952.

This genetic instability is strongly detrimental to the development of cell therapies, and in particular to the clinical applications of stem cells (Yamanaka, Pluripotent Stem Cell-Based Cell Therapy-Promise and Challenges. Cell stem cell. 27, 523-531 (2020); S. E. Peterson, J. F. Loring, Genomic instability in pluripotent stem cells: Implications for clinical applications. Journal of Biological Chemistry. 289, 4578-4584 (2014); K. Garber, RIKEN suspends first clinical trial involving induced pluripotent stem cells. Nature biotechnology. 33, 890-891 (2015)).

There is therefore a significant need for a solution for maintaining the genetic integrity of the cells in culture, in particular for large-scale production of cell therapies.

The aim of the invention is therefore to meet all of these needs and to overcome the disadvantages and limits of the prior art.

### SUMMARY OF THE INVENTION

By working on the development of cellular microcompartments for cell culture in 3D, the inventors developed a system allowing a cell mass culture while maintaining their genomic integrity.

For this purpose, the subject of the invention is a three-dimensional cellular microcompartment comprising at least one external hydrogel layer and inside said external layer at least one layer of cells and/or at least one cellular base layer, wherein less than 20% of the total population of cells present in the microcompartment are cells having at least one mutation, preferentially between 0 and 10%, even more preferentially between 0 and 5%, preferentially between 0 and 3%, even after several cell divisions.

According to another subject, the invention relates to an assembly of at least two three-dimensional cellular microcompartments, preferably in liquid suspension, each compartment comprising at least one external hydrogel layer and inside said external layer at least one layer of cells and/or at least one cellular base layer, in which less than 20% of the total population of cells present in all the microcompartments are cells having at least one mutation, preferentially between 0 and 10%, even more preferentially between 0 and 5%, in particular between 0 and 2%.

Advantageously, this level of mutant cells is lower than that of existing cell culture systems. For example, certain studies suggest that an inactivating mutation of the P53 gene confers, in a conventional 2D stem cell culture system, a selection advantage up to ×1.9 per pass (IPS-Cell Technology and the Problem of Genetic Instability—Can It Ever Be Safe for Clinical Use? Attwood & Edel)+Merkle, F. T.; Ghosh, S.; Kamitaki, N.; Mitchell, J.; Avior, Y.; Mello, C.; Kashin, S.; Mekhoubad, S.; Ilic, D.; Charlton, M.; et al. Human pluripotent stem cells recurrently acquire and expand dominant negative P53 mutations. Nature 2017, 545, 229-233.). This implies a 97% probability of attachment after emergence of this mutation (Haldane, J. B. S. A Mathematical Theory of Natural and Artificial Selection, Part V: Selection and Mutation. Math. Proc. Camb. Phills. Soc. 1927, 23, 838-844.)

Maintaining the genomic integrity of the cells makes it possible to use the microcompartments with the cell cultures according to the invention for different applications and in particular in the prevention and/or treatment of pathologies.

The cellular microcompartments according to the invention can be obtained in particular by the implementation of a specific preparation method comprising the following steps:
(a) preparing a suspension of cells comprising single cells and/or at least one cluster of cells in an isotonic medium, preferentially a culture medium containing an apoptosis inhibitor;
(b) encapsulating the cellular suspension in a hydrogel layer;
(c) culturing the resulting microcompartments in an isotonic solution, preferably a culture medium containing an apoptosis inhibitor;
(d) preferentially rinsing the microcompartments, so as to remove the apoptosis inhibitor;
(e) culturing the microcompartments for at least two cell division cycles (amplification); and
(f) optionally recovering the resulting cellular microcompartments;
the method being characterised in that all of the cells initially encapsulated in step (b) (at the time of encapsulation) represent a volume less than 50% of the volume of the microcompartment in which they are encapsulated.

This method makes it possible to obtain microcompartments according to the invention with a population of cells whose genomic integrity is kept and stabilised.

The invention also aims to use a cellular microcompartment and/or such a method for maintaining the genomic integrity of cells during their amplification.

Other features and advantages will emerge from the detailed description of the invention and the following examples.

### BRIEF DESCRIPTION OF THE FIGURES

FIG. 1 is an overview of the three experimental arms “2D culture,” “Bioreactor aggregates” and “Invention” as well as the numbering and chronology of the passes carried out (indicated in the rectangles D4=day 4, D8=day 8, etc.). All cultures were stopped at the final day, D28, for detailed genetic comparison.

FIG. 2a is a phase contrast microscopy image showing the results of the experimental arm “2D culture” at the final day, 28, before final sampling. 500 μm scale bar.

FIG. 2b is a phase contrast microscopy image showing the results of the experimental arm “Bioreactor aggregates” at the final day, 28, before final sampling. The aggregates shown were removed from their culture in suspension and temporarily deposited in a Petri dish to carry out the microscopic observation. 500 μm scale bar.

FIG. 2c is a phase contrast microscopy image showing the results of the experimental arm “Invention” at the final day 28 before final sampling. The microcompartments shown were removed from their culture in suspension and temporarily deposited in a Petri dish to carry out the microscopic observation. 500 μm scale bar.

FIG. 3 is a representation of the visible growth of the cells during the culture time calculated by counting the cells before and after each pass. The cumulative theoretical amplification factor is represented as a function of time; the axis of the ordinate (amplification) is shown on a logarithmic scale. The data points shown correspond to all the counts that were carried out at the time of the passes.

FIG. 4 is a representation of the results of the phenotypic evaluation of the stem cells by flow cytometry. The dissociated cells are fixed and labelled for the OCT4 and NANOG pluripotent markers. The percentage of double-positive cells for these 2 markers during successive passes for 28 days is represented here (average and standard deviation).

FIG. 5 is a high-resolution karyotype by Cytoscan HD array SNP chip for comparative analysis of the analysis of the initial day 0 sample and the 3 experimental arms: “2D culture,” “Bioreactor aggregates” and “Invention” at 28 days. CytoScan® HD Array Affymetrix, sold by ThermoFisher, quantifies the average number of copies per cell for 2.67 million probes distributed throughout the genome. The surrounded areas are centred on chromosome 20.

FIG. 6 shows the results of the evaluation by digital PCR of the average number of copies of the 20q11 chromosomal region during the 28 days of culture for the 3 experimental arms (Analysis carried out with the iPS ddPCR 24 probe test from Stemogenomics). On the left, count of 20q11 copies as a function of the number of days in culture. On the right, count of 20q11 copies ratio to the cumulative theoretical amplification over time. The data points correspond to the samplings carried out during the various passes: Squares=“Bioreactor aggregates,” Circles=“2D culture” and Triangles=“Invention.” The associated curves correspond to the corresponding regressions. It should be noted that the standard deviations for these measurements are on average 0.12 (count of 20q11 copies), the stars indicate the measurements which are significantly increased.

FIG. 7 shows the summary of the percentages of mutated cells during 28 days of culture for the “2D culture,” “Bioreactor aggregates” and “Invention” arms for example 1.

FIG. 8 shows the karyotype, obtained by digital PCR, of the two cell lines (GHE and AAVS1_GFP) used in example 2.

FIG. 9 is an overview of the two experimental arms “Bioreactor aggregates” and “Invention” and also the numbering and chronology of the passes made (indicated in the rectangles d4=day 4, d8=day 8, etc.).

FIGS. 10A and 10B are phase contrast microscopy images showing the results of the experimental arms A: “Bioreactor aggregates” and B: “Invention” on day 19 for “Bioreactor aggregates” and on day 21 for “Invention.” The aggregates as well as the microcompartments shown were removed from their culture in suspension and temporarily deposited in a Petri dish to carry out the microscopic observation. 500 μm scale bar.

FIG. 11 is a representation of the visible growth of the cells during the culture time calculated by counting the cells before and after each pass. The cumulative theoretical amplification factor is represented as a function of time; the axis of the ordinate (amplification) is shown on a logarithmic scale. The data points shown correspond to all the counts that were carried out at the time of the passes.

FIG. 12 is a representation of the results of the phenotypic evaluation of the stem cells by flow cytometry. The dissociated cells are fixed and labelled for the OCT4 and NANOG pluripotent markers. The percentage of double-positive cells for these 2 markers during successive passes for 28 days is represented here (average and standard deviation).

FIG. 13 shows the results of the evaluation by digital PCR of the percentage of GFP− (iPSC-GHE) and GPFP+ (IPSC-AAVS1) cells during the 21 days of culture for the 2 experimental arms. On the left: percentage of GFP− and GFP+ cells as a function of the number of days in culture. On the right: percentage of GFP− and GFP+ cells relative to the cumulative theoretical amplification over time. The data points correspond to the samplings carried out during the various passes: Squares=“Bioreactor aggregates” and Triangles=“Invention.”

FIG. 14 shows the results of the evaluation by digital PCR of the average count of copies of the 7q and 20q chromosomal region during the 28 days of culture for the 2 experimental arms (Analysis carried out with the iPS ddPCR 24 probe test from Stemogenomics). On the left, count of copies 7q and 20q as a function of the number of days in culture. On the right, count of 7q and 20q copies ratio to the cumulative theoretical amplification over time. The data points correspond to the samplings carried out during the various passes: Squares=“Bioreactor aggregates” and Triangles=“Invention.”

FIG. 15 shows the summary of the percentages of mutated cells during 28 days of culture for the “Bioreactor aggregates” and “Invention” for arms example 2.

### DETAILED DESCRIPTION OF THE INVENTION

### Definitions

For the purposes of the invention, “alginate” means linear polysaccharides formed from β-D-mannuronate and α-L-guluronate, salts and derivatives thereof.

For the purposes of the invention, “hydrogel capsule” means a three-dimensional structure formed from a matrix of polymer chains, swollen using a liquid, preferentially water.

For the purposes of the invention, “cell expressing a gene” means a cell which contains at least 5 times more copies of the RNA transcribed from the DNA sequence of the gene in question compared to a pluripotent cell, preferentially 10 times more copies, preferentially 20 times more copies, preferentially 100 times more copies.

For the purposes of the invention, “differentiated” cells means cells which have a particular phenotype, as opposed to pluripotent stem cells which are not differentiated or progenitor cells which are undergoing differentiation.

For the purposes of the invention, “human cells” means human cells or immunologically humanized non-human mammalian cells. Even when this is not specified, the cells, stem cells, progenitor cells and tissues according to the invention consist of or are obtained from human cells or from immunologically humanized non-human mammalian cells.

For the purposes of the invention, the term “mutant cell” refers to a cell carrying at least one mutation.

For the purposes of the invention, “progenitor cell” means a stem cell that is already engaged in cell differentiation but that has not yet differentiated.

For the purposes of the invention, “embryonic stem cell” means a pluripotent stem cell of cells derived from the internal cell mass of the blastocyst. The pluripotency of the embryonic stem cells can be evaluated by the presence of markers such as the transcription factors OCT4, NANOG and SOX2 and surface markers such as SSEA3/4, Tra-1-60 and Tra-1-81. The embryonic stem cells used in the context of the invention are obtained without destroying the embryo from which they originate, for example using the technique described in Chang et al. (Cell Stem Cell, 2008, 2(2): 113-117). Optionally, human embryonic stem cells can be excluded from the invention and in this case the subject matter of the invention excludes human embryonic stem cells.

For the purposes of the invention, “pluripotent stem cell” or “pluripotent cell” means a cell which has the capacity to form all the tissues present in the entire organism of origin, without however being able to form an entire organism per se. Human pluripotent stem cells can be called hPSC in the present application. These may in particular be induced pluripotent stem cells (iPSC or hiPSC for human induced pluripotent stem cells), embryonic stem cells or MUSE cells (for “multilineage-differentiating stress enduring”).

For the purposes of the invention, “induced pluripotent stem cell” means a pluripotent stem cell induced to become pluripotent by genetic reprogramming of differentiated somatic cells. These cells are in particular positive for pluripotency markers, such as staining with alkaline phosphatase and expression of the proteins NANOG, SOX2, OCT4 and SSEA3/4. Examples of methods for obtaining induced pluripotent stem cells are described in the articles by Yu et al. (Science 2007, 318 (5858): 1917-1920), Takahashi et al (Cell, 207, 131(5): 861-872) and Nakagawa et al (Nat Biotechnol, 2008, 26(1): 101-106).

The “Feret diameter” of a microcompartment according to the invention means the distance “d” between two tangents to said microcompartment, these two tangents being parallel, such that the entire projection of said microcompartment is comprised between these two parallel tangents.

For the purposes of the invention, “variable thickness” of the inner layer of human cells undergoing cell differentiation means that, in the same microcompartment, the inner layer does not have the same thickness throughout.

For the purposes of the invention, “microcompartment” or “capsule” means a partially or entirely closed three-dimensional structure containing several cells.

For the purposes of the invention, “convective culture medium” means a culture medium stirred by internal movements.

For the purposes of the invention, the term “mutation” means a genetic or epigenetic mutation, preferentially a functional mutation. It may in particular involve a point modification of the genetic sequence, a structural variant, an epigenetic modification, or a modification of the mitochondrial DNA. It may involve a mutation by amplification of a chromosomal region, such as for example a mutation by amplification of the 20q chromosomal region, in particular 20q11, or alternatively 7q.

The term “functional mutation” within the meaning of the invention refers to a transmissible genetic or epigenetic modification which confers a potential gain or loss of function or loss of potential function to a relevant mutant cell. It preferably involves a mutation causing a modification of the phenotype of the affected mutant cell. Very preferentially, it involves a change of the genomic and/or of the epigenomic sequence which alters the therapeutic potential of a population of cells, or by increasing the risk associated with the therapy produced, or by decreasing the benefit provided by the therapy produced.

The “largest dimension” of a microcompartment or a cell cluster or of a layer of cells or of a cellular base layer within the meaning of the invention is understood to mean the value of the largest Feret diameter of said microcompartment.

The “smallest dimension” of a microcompartment or a cell cluster or of a layer of cells or of a cellular base layer within the meaning of the invention is understood to mean the value of the smallest Feret diameter of said microcompartment.

For the purposes of the invention, “tissue” or “biological tissue” has the common meaning for tissue in biology, that is to say, the intermediate organisation level between cell and organ. A tissue is an assembly of similar cells and of the same origin (most commonly derived from a common cell line, although they can originate in the association of distinct cell lines), grouped into a cluster, network or bundle (fibre). A tissue forms a functional assembly, that is to say that its cells contribute to the same function. Biological tissues regenerate regularly and are assembled together to form organs.

For the purposes of the invention, “lumen” means a volume of aqueous solution topologically surrounded by cells. Preferentially, its content is not in diffusive equilibrium with the volume of convective liquid present outside the microcompartment.

### Cellular Microcompartments

The subject of the invention is a three-dimensional cellular microcompartment comprising at least one external hydrogel layer and inside said external layer at least one layer of cells and/or at least one cellular base layer, wherein less than 20% of the total population of cells present are cells having at least one mutation.

The microcompartment comprises an external hydrogel layer. Preferentially, the hydrogel used is biocompatible, that is to say it is non-toxic to the cells. The hydrogel layer must allow the diffusion of oxygen and nutrients in order to supply the cells contained in the microcompartment and to enable them to survive. According to one embodiment, the external hydrogel layer comprises at least alginate. It may consist exclusively of alginate. The alginate can in particular be a sodium alginate, composed of 80% α-L-guluronate and 20% β-D-mannuronate, with an average molecular weight of 100 to 400 kDa and a total concentration of between 0.5 and 5% by weight. The hydrogel layer has no cells.

The hydrogel layer makes it possible in particular to protect the cells from the outside environment and to limit the uncontrolled proliferation of the cells.

The microcompartment according to the invention comprises at least one layer of cells and/or at least one cellular base layer. This or these layer(s) of cells and/or cellular base layer(s) is (are) organised three-dimensionally in the microcompartment.

The microcompartment may in particular comprise:
one or more layers of cells and/or one or more cellular base layers, organised three-dimensionally, or
one or more layers of cells and/or one or more one or more cellular base layers, organised three-dimensionally, and cells in suspension in the microcompartment.

The cells present in the microcompartment may be any cell type. Preferably, the cells are human or animal cells.

In a particular embodiment, the microcompartment comprises pluripotent stem cells. A pluripotent stem cell, or pluripotent cell, refers to a cell that has the ability to form all the tissues present in the whole organism of origin, without being able to form an entire organism as such. The pluripotent stem cells can in particular be induced pluripotent stem cells (IPS), MUSE cells (“Multilineage-differentiating Stress Enduring”) that are found in skin and bone marrow of adult mammals, or embryonic stem cells (ES).

According to a particularly suitable variant of the invention, the microcompartment according to the invention comprises human or animal induced pluripotent stem cells.

In another particular embodiment, the microcompartment according to the invention comprises human or animal multipotent cells and/or human or animal progenitor cells derived from these multipotent cells. The multipotent and/or progenitor cells were preferentially obtained from pluripotent stem cells, in particular human pluripotent stem cells, or optionally from non-pluripotent human cells, the transcriptional profile of which was artificially modified to match that of specific multipotent and/or progenitor cells, typically by forced expression of specific transcription factors for the target cellular phenotype. Preferentially, the multipotent and/or progenitor cells were obtained from pluripotent stem cells after bring into contact with a solution capable of initiating the differentiation of said stem cells.

According to another variant, the microcompartment according to the invention comprises human or animal differentiated cells. The differentiated cells were preferably obtained from pluripotent stem cells or progenitor cells, in particular human pluripotent stem cells or human progenitor cells, or optionally from non-pluripotent human cells whose transcriptional profile was artificially modified to join that of particular differentiated cells, typically by forced expression of specific transcription factors of the target cell phenotype. Preferentially, the differentiated cells were obtained from pluripotent or multipotent, or progenitor stem cells after bringing into to contacting with a solution capable of initiating differentiation of said stem cells. According to one variant, the cellular content of the microcompartment comprises homogeneous or mixed cell identities.

The differentiated cells may in particular be in the form of a three-dimensional tissue or micro-tissue or in the form of a plurality of fabric or micro-tissues in the microcompartment. It may be a compacted tissue or micro-tissue.

The microcompartment according to the invention may comprise several types of cells. In particular, a microcompartment according to the invention may comprise, for example, stem cells with induced pluripotency and/or multipotent cells and/or progenitor cells and/or differentiated cells.

If the cells encapsulated in the microcompartment are intended to be used in cell therapy in human beings, the cells can be immuno-compatible with the person intended to receive them in order to avoid any risk of rejection.

The cells present in the microcompartment are carrying little, or even no functional mutation. According to the invention less than 20% of the total population of cells present are cells having at least one mutation, in particular at least one functional, genetic or epigenetic mutation.

The invention targets in particular the microcompartments in which less than 20% of the total population of cells present are cells having at least one functional mutation, preferentially the microcompartments in which less than 20% of the total population of cells present are cells having at least one mutation leading to a modification of the phenotype of the mutant cell concerned.

The invention also targets the microcompartments in which less than 20% of the total population of cells present are cells having at least one mutation allowing clonal expansion of cells which becomes dominant in culture.

According to a particularly suitable variant, the invention relates to a microcompartment in which less than 20% of the total cell population present are cells having at least one mutation selected from oncogenic mutations. At least one mutation is an oncogenic mutation.

According to an embodiment, the invention relates to a microcompartment in which less than 20% of the total cell population present are cells having at least one mutation of a gene and/or a mutation by amplification of a chromosomal region.

In one embodiment of the invention, less than 20% of the total cell population present in the microcompartment are cells having at least one mutation of the P53 gene and/or an amplification of the 20q and/or 7q chromosomal region (mutation by amplification of the 20q and/or 7q chromosomal region), in particular an amplification of the 20q11 chromosomal region (mutation by amplification of the 20q11 chromosomal region).

Preferably, the cells having at least one mutation according to one of the embodiments of the invention represent between 0 and 15% of the total cell population present in the microcompartment, in particular between 0 and 14%, between 0 and 12%, in particular between 0 and 10%, even more preferentially between 0 and 8%, between 0 and 5%, between 0 and 2%.

The percentage of mutant cells among a cell population can be measured by various methods known to a person skilled in the art. For the detection of point mutations, sequencing methods with a high reading depth are preferred (Whole Genome sequencing, Exome sequencing, Amplitude, etc.). For the detection of the structural variants, high resolution methods are preferred (High resolution SNP array, Bionano optical genome mapping, digital PCR, etc.). For the detection of the epigenetic variants several tools can be envisaged (RRBS methylation arrays, bisulfite sequencing/pyrosequencing, etc.).

Advantageously, the microcompartments according to the invention have a very low level of mutant cells, and this is so after several cell division cycles. The cells according to the invention are indeed cells obtained by amplification, from at least one cell.

In fact, the cells present in the microcompartment according to the invention were obtained after at least two cell division cycles after encapsulation in an external hydrogel layer of at least one cell.

Preferably, the cells present in the microcompartment according to the invention were obtained after at least 2, 3, 4, 5, 6, 7, 8, 9, 10, 11, 12, 13, 14, 15, 20, 25, 28, 30 cell division cycles after encapsulation in an external hydrogel layer of at least 1 cell, preferentially between 1 and 5, between 1 and 10, between 1 and 15, between 1 and 20, between 1 and 30, between 1 and 40, between 1 and 50, between 1 and 60, between 1 and 100 cells. For example, the cells present in the microcompartment were obtained after at least six cell division cycles after encapsulation in an external hydrogel layer of at least 1 cell, preferentially between 1 and 50 cells.

Preferably, the microcompartment is obtained after at least 2 passes after encapsulation, more preferably at least 3, 4, 5, 6, 7, 8, 9 or 10 passes. Each pass may last for example between 2 and 15 days, in particular between 3 and 10 days.

Preferably, the microcompartment is obtained after at least one re-encapsulation, more preferably between 1 and 14 re-encapsulations, in particular between 2 and 7 re-encapsulations. Very preferentially, a re-encapsulation corresponds to a new pass and each encapsulation cycle corresponds to a pass.

Preferably, all of the cells initially encapsulated in the microcompartment before the first cell division cycle represents a volume less than 50% of the volume of the microcompartment in which they are encapsulated, more preferably less than 40%, 30%, 20%, 10% of the volume of the microcompartment in which they are encapsulated.

Thus, according to one embodiment, the cells present in the microcompartment according to the invention were obtained after at least 2, 3, 4, 5, 6, 7, 8, 9, 10, 11, 12, 13, 14, 15, 20, 25, 28, 30 cell division cycles, after encapsulation in an external hydrogel layer of cell(s) representing a volume less than 50% of the volume of the microcompartment in which they are encapsulated, more preferably less than 40%, 30%, 20%, 10% of the volume of the microcompartment in which they are encapsulated.

Preferably, in the microcompartment according to the invention, the cells represent more than 50% by volume relative to the volume of the microcompartment, even more preferably more than 60%, 70%, 75%, 80%, 85%, 90% by volume relative to the volume of the microcompartment.

The microcompartment according to the invention comprises several cells, preferably at least 20 cells, even more preferably at least 100, at least 500, at least 1000, at least 10,000.

In addition to the external layer and cells, the microcompartment according to the invention may comprise other elements, in particular:
a culture medium, and/or
at least one intermediate layer of isotonic aqueous solution and/or extracellular matrix elements.

The culture medium is a medium suitable for the cells present in the microcompartment according to the knowledge of a person skilled in the art.

The intermediate layer of isotonic aqueous solution preferentially contains extracellular matrix elements, such as peptide or peptidomimetic sequences capable of binding to integrins. “Isotonic aqueous solution” means an aqueous solution having an osmolarity of between 200 and 400 mOsm/L. This layer is preferably situated between (a) the layer(s) of cells and/or the cellular base layers(s) and (b) the external hydrogel layer.

The intermediate layer may consist of elements which have been added during the production of the microcompartment and/or of elements added to the microcompartment and/or of elements secreted or induced by the other constituents of the microcompartment.

The intermediate layer may in particular comprise or consist of an extracellular matrix and/or a culture medium. If it comprises extracellular matrix, this may be extracellular matrix secreted by cells of the inner layer and/or by extracellular matrix added at the time of the preparation/production of the microcompartment.

The intermediate layer preferentially comprises a mixture of proteins and extracellular compounds necessary for culturing cells undergoing differentiation. Preferentially, the intermediate layer comprises structural proteins, such as collagen, laminins, entactin, vitronectin, and growth factors, such as TGF-beta and/or EGF. According to one variant, the intermediate layer may consist of or comprise Matrigel® and/or Geltrex® and/or a hydrogel-type matrix of plant origin such as modified alginates or of synthetic origin or copolymer of poly(N-isopropylacrylamide) and poly(ethylene glycol) (PNIPAAm-PEG), of Mebiol® type.

According to one variant, the intermediate layer may form a gel.

At the surface of the intermediate layer in contact with the inner layer of human cells undergoing differentiation, the intermediate layer may optionally contain one or more cells.

Preferably, the intermediate layer has a Young's modulus of between 0.05 and 3 kDa. The Young's modulus can be measured by any method known to a person skilled in the art, in particular by measuring the rheology of gels of the same composition as the intermediate layer or else by AFM (atomic force microscopy).

An intermediate layer of isotonic aqueous solution and/or comprising extracellular matrix elements, preferentially an intermediate layer of extracellular matrix, with such values of Young's modulus make it possible to improve the maintenance of the cell phenotype and the genomic integrity of the cells contained in this intermediate layer during cell divisions.

According to a particular embodiment of the invention, the microcompartment also comprises at least one opening or lumen. Preferably, the microcompartment comprises an internal lumen. The microcompartment according to the invention could also comprise several lumens. The lumen(s) may contain a liquid, in particular the culture medium and/or a liquid secreted by the cells. Advantageously, the presence of this hollow part enables the cells to have a small diffusive volume of which they can control the composition, promoting cellular communication.

In one variant of the invention, the microcompartment successively comprises, organised around a lumen:
at least one layer of cells and/or at least one cellular base layer, preferentially of epithelial cells, in particular stem cells and in particular human or animal induced pluripotent stem cells.
an intermediate layer of isotonic aqueous solution and/or extracellular matrix elements, preferentially an extracellular matrix layer;
an external hydrogel layer.

In this variant the inner layer of cells within the microcompartment according to the invention is hollow. This three-dimensional arrangement in a single layer or spherical epithelial base surrounding a central lumen may also be called a cyst. The lumen(s) are preferably generated, at the time of the formation of the cyst, by the cells which multiply and develop on the extracellular matrix layer.

The cyst-shaped conformation it makes it possible to reduce the pressures experienced by the stem cells relative to the 2D cultures or aggregates. This configuration reduces cell mortality, increasing the culture amplification factor. Consequently, this makes it possible to reduce the number of necessary passes and dissociation to reduce the culture time necessary to reach the necessary final number of cells. Collectively these improvements also participate in maintaining the genetic integrity of the stem cells in the microcompartments.

The cellular microcompartment according to the invention is closed or partially closed, that is to say that the external layer is closed or partially closed. Preferentially, the microcompartment is closed.

The microcompartment according to the invention can be in any three-dimensional form, that is, it may have the shape of any object in space. The microcompartment may have any form compatible with cell encapsulation. Preferentially, the microcompartment according to the invention is in spherical or elongated form. It may have the shape of an ovoid, a cylinder, a spheroid or a sphere. It may in particular be in the form of a hollow spheroid, a hollow ovoid, a hollow cylinder or a hollow sphere.

It is the outer layer of the microcompartment, that is the hydrogel layer, which imparts its size and shape to the microcompartment according to the invention. Preferentially, the smallest dimension of the microcompartment according to the invention is between 10 μm and 1 mm, preferentially between 100 μm and 700 μm. It may be between 10 μm and 600 μm, in particular between 10 μm and 500 μm.

Its largest dimension is preferentially greater than 10 μm, more preferentially between 10 μm and 1 m, even more preferentially between 10 μm and 50 cm.

The microcompartment according to the invention contains cells whose genomic integrity was preserved and/or maintained, a very small percentage of cells present in the microcompartment being carriers of mutations. It can be used for any application, in particular as a drug in cell therapy in humans or animals.

The microcompartment according to the invention may optionally be frozen to be stored. It will then have to be thawed before it is used.

The invention also relates to a plurality of microcompartments together.

Thus, the invention also relates to an assembly or a series of cellular microcompartments as described above comprising at least two cellular microcompartments according to the invention.

The invention also relates to an assembly or a series of microcompartments of at least two three-dimensional cellular microcompartments, each microcompartment comprising at least one external hydrogel layer and inside said external layer at least one layer of cells and/or at least one cellular base layer, wherein at least one microcompartment is a microcompartment according to the invention.

Another particular object of the invention is an assembly or series of at least two three-dimensional cellular microcompartments, each microcompartment comprising at least one external hydrogel layer and inside said external layer at least one layer of cells and/or at least one cellular base layer, wherein less than 20% of the total population of cells present in all the microcompartments of the assembly are cells having at least one mutation. Preferably, the cells having at least one mutation represent between 0 and 15% of the total population of cells present in all the microcompartments, in particular between 0 and 14%, between 0 and 12%, in particular between 0 and 10%, even more preferentially between 0 and 8%, between 0 and 5%, between 0 and 2%. Preferably, at least one microcompartment is a microcompartment according to the invention.

Thus one or more microcompartments of the series may comprise more than 20% by number of mutant cells relative to the number of cells present in said microcompartment(s), but for all the microcompartments forming the microcompartment assembly according to the invention, less than 20% of the total population of cells present in all the microcompartments of the assembly are cells having at least one mutation, in particular at least one functional, genetic or epigenetic mutation. Preferably, at least one microcompartment is a microcompartment according to the invention.

The invention relates in particular to a microcompartment assembly in which less than 20% of the total population of cells present in the assembly are cells having at least one functional mutation, preferably a microcompartment assembly in which less than 20% of the total population of cells present in the assembly are cells having at least one mutation causing a modification of the phenotype of the mutant cell concerned.

The invention also relates to a microcompartment assembly in which less than 20% of the total population of cells present in the assembly are cells having at least one mutation allowing clonal expansion of the cells which becomes dominant in culture.

According to a particularly suitable variant, the invention relates to a microcompartment assembly wherein less than 20% of the total population of cells present in the assembly are cells having at least one mutation selected from oncogenic mutations. At least one mutation is an oncogenic mutation.

According to one embodiment, the invention relates to a microcompartment assembly wherein less than 20% of the total population of cells present in the assembly are cells having at least one mutation of a gene and/or a mutation by amplification of a chromosomal region.

In one embodiment of the invention, less than 20% of the total cell population present in the microcompartment assembly are cells having at least one mutation of the P53 gene and/or an amplification of the 20q and/or 7q chromosomal region (mutation by amplification of the 20q and/or 7q chromosomal region), in particular an amplification of the 20q11 chromosomal region (mutation by amplification of the 20q11 chromosomal region).

Preferably, the cells having at least one mutation according to one of the embodiments of the invention, represent between 0 and 15% of the total cell population present in the microcompartment assembly, in particular between 0 and 14%, between 0 and 12%, in particular between 0 and 10%, even more preferentially between 0 and 8%, between 0 and 5%, between 0 and 2%.

Preferably, the cells present in the microcompartments of the microcompartment assembly according to the invention were obtained after at least 2, 3, 4, 5, 6, 7, 8, 9, 10, 11, 12, 13, 14, 15, 20, 25, 28, 30 cell division cycles after encapsulation in an external hydrogel layer of at least 1 cell per microcompartment. The microcompartment (s) present in this microcompartment assembly may have one or more characteristics of a microcompartment according to the invention (size, shape, cell count, volume of cells, intermediate layer, lumen, etc.).

The microcompartment assembly according to the invention preferably comprises between 2 and 1016 microcompartments.

Preferably, the series of microcompartments according to the invention is in a culture medium, in particular in an at least partially convective culture medium.

According to a particularly suitable embodiment, the object of the invention is a series of cellular microcompartments as described above in a closed chamber, such as a bioreactor, preferentially in a culture medium in a closed chamber, such as a bioreactor.

The presence of an external hydrogel layer and possibly an intermediate layer of isotonic aqueous solution enables uniform distribution of the cells between the microcompartments. Moreover, this hydrogel layer makes it possible to prevent microcompartments from merging, these merger events being a major source of variability which is unfavourable for phenotypic homogeneity of cells.

### Method for Obtaining a Microcompartment According to the Invention

The invention also relates to a method for preparing microcompartments according to the invention.

The method for preparing a microcompartment or a microcompartment assembly according to the invention may comprise at least the implementation of the steps which consist of:
(a) preparing a suspension of cells comprising single cells and/or at least one assembly of cells in an isotonic medium, preferentially a culture medium containing an apoptosis inhibitor;
(b) encapsulating the cellular suspension in a hydrogel layer;
(c) preferentially culturing the resulting microcompartments in an isotonic solution, preferably a culture medium containing an apoptosis inhibitor;
(d) preferentially rinsing the microcompartments, so as to remove the apoptosis inhibitor;
(e) culturing the microcompartments in an isotonic solution, preferentially a culture medium, for at least two cell division cycles; and
(f) optionally recovering the resulting cellular microcompartments.

The invention also aims to use this method to maintain the genomic integrity of the encapsulated cells.

In the method according to the invention, all of the cells initially encapsulated in step (b) represent a volume less than 50% of the volume of the microcompartment in which they are encapsulated, more preferably less than 40%, 30%, 20%, 10% of the volume of the microcompartment in which they are encapsulated.

The apoptosis inhibitor may for example be one or more inhibitor(s) of RHO/ROCK (RHO-associated protein kinase) pathways, or any other apoptosis inhibitor known to the person skilled in the art. The apoptosis inhibitor must make it possible to promote cell survival, and in the case of the presence of an extracellular matrix, the adhesion of the cells to the extracellular matrix at the time of formation of the external hydrogel layer around said extracellular matrix.

The method according to the invention may comprise before or simultaneously with step (a) a step of dissociation of the cells by chemical, enzymatic or mechanical dissociation. This step is particularly important in the case of adherent cells.

The encapsulated cells are suspended in the form of single cells and/or clusters or assembly(ies) of at least two cells (“cluster(s)”). Preferably, the single cells represent less than 50% by number of all the cells initially encapsulated in step (b). Indeed, it is preferable to encapsulate clusters of cells because this reduces the chromosomal desegregation and consequently reduces the appearance of new mutations and participates in maintaining the genomic integrity of the cells.

Preferably, each cell cluster initially encapsulated in step (b) has a larger dimension less than 20% of the largest dimension of a microcompartment in which it is encapsulated, even more preferentially less than 10%. In fact, the cell clusters must not have a size that is too large compared to the size of the microcompartment since a dimension of these initial cell clusters that is too large could lead, during cell divisions, to a cell confluence in the capsule that is too early; this too early confluence of all or part of the capsules could lead to an increase in intracellular pressures and lead to cellular stress, affecting in particular chromosomal segregation.

According to one variant, the method according to the invention may comprise a step of mixing the cells with an extracellular matrix, either between step (a) and step (b), or simultaneously with the encapsulation in step (b).

Very preferably, steps (c), (d) and (e) are carried out under continuous or sequential stirring. This stirring is important because it maintains the homogeneity of the culture environment and avoids the formation of any diffusion gradient. For example, it allows homogeneous control of cellular oxygenation level; thus avoiding hypoxia-related necrosis or hyperoxia-related oxidative stress phenomena. By avoiding an increase in cell mortality and/or oxidative stress, stirring participates in maintaining the genetic integrity.

The method according to the invention is preferably implemented in a closed chamber such as an enclosed bioreactor.

The number of cell divisions in step (e) is at least 2, 3, 4, 5, 6, 7, 8, 9, 10, 11, 12, 13, 14, 15 cell division cycles.

Preferably, the microcompartment is obtained after at least 2 passes (here a pass corresponds to a complete cycle of steps (a), (b), and (e), optionally (c) and (d)), more preferably at least 3, 4, 5, 6, 7, 8, 9 or 10 passes. Each pass may last for example between 2 and 15 days, in particular between 3 and 8 days.

In a preferred variant, the method according to the invention comprises at least one re-encapsulation of the cells after step (e), that is at least two encapsulation cycles. Preferably, each encapsulation cycle corresponds to a pass. In this variant of the method (at least one re-encapsulation of the cells after step (e)), the number of cell divisions of the entire method (for all passes) is at least 3, 4, 5, 6, 7, 8, 9, 10, 11, 12, 13, 14, 15, 20, 25, 30 cell division cycles.

In a method according to the invention, there may be several re-encapsulations, preferably between 1 and 100, in particular between 1 and 10 re-encapsulation(s).

Each re-encapsulation can comprise:
a step which consists of dissociating the microcompartment or the series of microcompartments in order to obtain a suspension of cells or a suspension of cell clusters; the external hydrogel layer may be eliminated in particular by hydrolysis, dissolution, piercing and/or breaking by any biocompatible means, that is to say means which are not toxic for the cells. For example, the elimination may be accomplished using phosphate-buffered saline, a divalent ion chelator, an enzyme such as alginate lyase if the hydrogel comprises alginate, and/or laser microdissection, and
a step of re-encapsulating all or part of the cells or cell clusters in a hydrogel capsule.

Re-encapsulation is a means suitable for increasing the cell amplification resulting from the pluripotent step, and reducing the risks of mutation.

According to one embodiment, the re-encapsulation comprises the following steps:
(i) removing the external hydrogel layer,
(ii) resuspending the cells that were contained in the microcompartment so as to obtain single cells and/or at least one assembly or cluster of cells in an isotonic medium, preferentially a culture medium containing an apoptosis inhibitor,
(iii) encapsulating the cellular suspension in a hydrogel layer;
(iv) preferentially culturing the resulting microcompartments in an isotonic solution containing an apoptosis inhibitor, preferably a culture medium containing an apoptosis inhibitor;
(v) preferentially rinsing the microcompartments, so as to remove the apoptosis inhibitor;
(vi) culturing the microcompartments in an isotonic solution, preferentially a culture medium, for at least one cell division cycle; and
(vii) optionally recovering the resulting cellular microcompartments.

The compartmentalisation in microcompartments makes it possible to eliminate the microcompartments containing even more mutated cells than the other capsules. Even if the mutated cells have a rapid growth they will reach the capsular confluence which will limit their multiplication. The compartmentalisation also makes it possible not to contaminate the entire cell population, and also to eliminate the capsules containing mutant cells, at any time, in particular before a re-encapsulation step. This sorting may be done either by inline analysis, or by eliminating filled capsules more quickly than others, for example. Thus, the method according to the invention may comprise one or more steps of removing the microcompartments comprising mutant cells, in particular microcompartments comprising more than 20% mutant cells.

According to one variant of the invention, the cells are pluripotent stem cells organised into cysts directly from pluripotent stem cells, or from differentiated cells which will be reprogrammed into pluripotent cells inside the hydrogel capsule during the formation of the microcompartments.

The incubation of steps (a) and/or (ii) is carried out preferentially for a time included between a few minutes and a few hours, preferentially between 2 minutes and 2 hours, more preferably between 10 minutes and 1 hour.

Step (c) and/or (iv) of culturing with an apoptosis inhibitor is carried out for a time included between 2 and 72 hours, preferentially for a time included between 6 and 48 hours, more preferably for a time comprised between 24 and 48 hours.

The rinsing step can be carried out by one or more rinsing operations, in successive culture media free of RHO/ROCK pathway inhibitors, less than 96 hours, preferentially less than 72 hours, more preferably between 24 and 48 hours after the start of steps (c) and/or (iv).

In one embodiment, at least one of the steps (preferably all the steps) is carried out at a temperature suited to the survival of the cells, included between 4 and 42° C. The temperature during cell proliferation must preferably be between 32 and 37° C. to avoid triggering mutations by lowering the performance of the repair enzymes. Likewise, preferably, the temperature must be low (ideally about 4° C.) to manage the stress on the cells in step (b).

According to one variant, the cell reprogramming agents can be added in step (a) and/or (b) and/or (c) and/or (ii) and/or (iii) and/or (iv). Preferably, they are cell reprogramming agents that do not permeate the hydrogel layer. The addition of reprogramming agents is particularly relevant when the initially encapsulated cells are differentiated cells that are to be dedifferentiated in particular up to the pluripotent stage. A person skilled in the art knows how to reprogramme a differentiated cell into a stem cell by reactivating the expression of genes associated with the embryonic stage by means of specific factors, designated in the present invention as “reprogramming agents.” The methods described in the following may be cited as examples: Takahashi et al., 2006 (“Induction of pluripotent stem cells from mouse embryonic and adult fibroblast cultures by defined factors” Cell, 2006 Vol 126, pages 663-676), Ban et al., 2009 (“Efficient induction of transgene-free human pluripotent stem cells using a vector based on Sendai virus, an RNA virus that does not integrate into the host genome” Proc Jpn Acad Ser B Phys Biol Sci. 2009; 85(8):348-62) and the international application WO2010/105311 whose title is “Production of reprogrammed pluripotent cells.” The reprogramming agents are advantageously co-encapsulated with the differentiated cells, so as to concentrate the product and promote the contact with all of the cells. In the case of reprogramming agents permeating the hydrogel layer, it is possible to add said agents to the culture medium after the encapsulation step. Reprogramming agents make it possible to impose a succession of phenotypic changes on the cells to the pluripotent stage. Advantageously, the step of reprogramming is carried out using specific culture media, promoting these phenotypic changes. For example, the cells are cultured in a first medium comprising 10% of human, or bovine serum, in an Eagle's minimum essential medium (DMEM) supplemented with a serine/threonine protein kinase receptor inhibitor (such as the product SB-431542 (C₂₂H₁₆N₄O₃)), one or more RHO/ROCK (RHO-associated protein kinase) pathway inhibitors, such as thiazolin and/or Y-27632, fibroblast growth factors, such as FGF-2, ascorbic acid and antibiotics, such as Trichstan A (C₁₇H₂₂N₂O₃). Then the culture medium is replaced with medium promoting the multiplication of the pluripotent cells, such as the mTeSR®1 medium.

At any time, the method according to the invention may comprise a step consisting of verifying the phenotype of the cells contained in the microcompartment. This verification can be carried out by identifying the expression by at least part of the cells contained in the microcompartment, of at least one gene specific to the phenotype sought.

The cellular microcompartments obtained according to the methods of the invention can then be frozen before any use. The freezing is preferentially carried out at a temperature comprised between −190° C. and −80° C. The thawing can be carried out in a warm water bath (37 degrees preferentially) so that the cells thaw quite rapidly. The microcompartments according to the invention before they are used may be kept at more than 4° C. for a limited time before they are used, preferentially between 4° C. and 38° C.

The method according to the invention, with its particular features, makes it possible to maintain the genomic integrity of the cells during the culture, the final microcompartments having cells bearing little or no mutation.

In particular, the 3-dimensional structure of the cells in the microcompartment and the low percentage or even zero percentage of cells isolated during encapsulation (most of the cells being encapsulated in the form of a cluster of cells), reduces the chromosomal desegregation and consequently reduces the appearance of new mutations.

The invention also promotes amplification with a high amplification factor, which consequently reduces the culture time and the number of divisions to obtain a very large number of cells, and therefore limits mutagenesis.

The protection of the cells by virtue of the external layer and the presence of extracellular matrix elements when it is present reduces the chromosomal desegregation and limits the mechanical stress of the cells, and consequently reduces the appearance of new mutations.

The control of the culture parameters in a bioreactor also reduces oxidative stress, which participates in the reduction of new mutations.

Also, the subject of the invention is also the use of a method according to the invention for maintaining the genomic integrity of cells during their amplification.

The subject of the invention is also the use, to maintain the genomic integrity of cells during their amplification, of a three-dimensional microcompartment, preferably closed, preferentially of spherical or elongated shape, comprising at least one external hydrogel layer defining an inner part. Preferably, it involves the use of a cellular microcompartment according to the invention in its different variants as described in the present application. The invention also targets the use, to maintain the genomic integrity of cells during their amplification, an assembly of these microcompartments, preferentially in a closed bioreactor, even more preferentially a microcompartment assembly according to all the variants according to the invention and as described in the present application.

The invention is now illustrated by two examples and comparative results. These examples relate to the culture of human pluripotent stem cells, and more particularly human induced pluripotent stem (IPS) cells.

### Example 1

### Protocol:

The cell line used here, called iPS-IMGINE005, has previously been described in this publication: E. Quelennec, C. Banal, M. Hamlin, D. Clémantine, M. Michael, N. Lefort, Generation of two induced pluripotent stem cell lines IMAGINI004-A and IMAGINi005-A from healthy donors. Stem Cell Research, 101959 (2020).

The iPS line was generated according to the usual standards of the iPS culture in 2 dimensions. In order to monitor the virtually inevitable emergence of mutations during the prolonged culture of this line, a monitoring of the karyotype is carried out regularly (every 5 to 10 passes).

The starting point of the experiment conducted here is a frozen iPS cell sample, pass 2D number 23 post reprogramming. At this stage of culture and for this sample, the high-resolution karyotype tests were not capable of detecting an amplification of the 20q11 chromosomal region, but it was observed that a brief culture (fewer than 10 2D passes) of this sample leads to the emergence of a mutation by amplification of the 20q11 chromosomal region.

This cell starting point is particularly pertinent for testing the positive selection over time of a mutant clone in a population of cells in culture.

Indeed, the mutation by amplification of the 20q11 chromosomal region confers a growth advantage on the mutated clone; the greater the selection pressure of the culture system, the more the risk this clone will be rapidly selected and become predominant.

An encapsulated culture system in stirred suspension (hereinafter referred to as “Invention”) was compared to two culture systems standard in the field of the production of pluripotent stem cells: culture in 2 dimensions (hereinafter referred to as “2D culture”) and the culture in an unprotected stirred suspension in the form of aggregates (hereinafter referred to as “Bioreactor aggregates”).

The initial sample (previously described and cultivated in 2D) was used to initiate 3 experimental arms in parallel associated with the 3 culture systems, and this was done over a period of 28 days. On each pass, and for each experimental arm, cells are sampled to allow the performance of genetic tests (See the results section). In particular, the frequency of the mutation by amplification of the 20q11 chromosomal region is evaluated at the start and at the end of this prolonged culture of 28 days.

The rate of the passes for each culture system scrupulously follows the optimal recommendations for each condition. Thus, the 2D cultures are passed every 4 to 5 days when the confluence is between 70 and 90%; the aggregate cultures are passed every 5 days according to the supplier's recommendations (Minibio, ABLE® Bioreactor Systems); the encapsulated cultures are passed every 7 days when the average capsular confluence is between 50 and 100%.

All the cultures described below are carried out with the mTeSR 1 culture medium (“Stemcell Technologies”). A 10 μM Rock inhibitor treatment is initiated during the first 24 hours after a pass.

All the cultures (2D, Bioreactor aggregates and Invention) are maintained in a cell culture incubator at 37° C. and 5% CO₂.

The 2 experimental arms cultivating stem cells in suspension “Bioreactor aggregates” and “Invention” use Minibio, ABLE® Bioreactor Systems brand of 30 ml mini bioreactors; the constant stirring speed was set at 35 rotations per minute from the seeding until the cells are collected.

The 2 experimental arms culturing stem cells in three-dimensional cell grouping form in “Bioreactor aggregates” and “Invention” suspension use an enzymatic dissociation for successive passes: aggregates on the one hand and the encapsulated cysts on the other hand, are dissociated by use of a TryplE bath for 20 minutes at 37° C. The cells and small groupings (clusters) of cells resulting from this dissociation are then used to inoculate a new culture.

For cultures using an extracellular matrix, Matrigel (Corning) is used. Thus, for the 2D cultures, the flasks (T-Flak T75) are previously coated with matrigel; for the encapsulations or re-encapsulations, the cells are mixed with the matrigel before injection into the central microfluidic channel, the culture in aggregates does not require the use of extracellular matrix.

The “2D culture” is established in flasks (T-Flask T75) previously coated/carpeted with Matrigel®, the cell seeding concentration is between 10,000 and 30,000 cells per cm². The passes are carried out by the small aggregates method, by short use (less than 5 minutes) of a calcium chelator, RelesR (Stem cell technologies). The culture medium is completely changed on day 1 to remove the rock inhibitor (constant volume) treatment then daily.

The “Bioreactor aggregates” culture is initiated with the same cell suspension used to seed the “2D culture” and the “Invention” culture but with an initial concentration of 175,000 cells per ml of medium, for a total of 20 ml of medium. The culture medium is completely changed on day 1 to remove the rock inhibitor (constant volume) treatment, then 75% of the media is renewed daily (constant volume of 20 ml).

### Cultivation of the HiPSCs According to the Invention (Encapsulation According to the Invention):

Before encapsulation, the 2D stem cell colonies were detached using ReLeSR for 1 minute and then dissociated using Accutase (StemCell Technologies). The HiPSCs were then mixed in a 50/50 volume ratio with Matrigel at 4° C. to maintain the suspension in the liquid state. The final concentration of cells in the cell/matrix solution was therefore between 0.4 and 1.0×10⁶ viable cells/ml, called encapsulation density. Ethylene-tetrafluoroethylene (ETFE) tubes are connected to the three inlets of a 3D printed microfluidic colaminar flow device. A microcapillary tip made of extruded and polished glass (with a nozzle diameter of about 100 μm for most experiments or a nozzle diameter of 150 μm) is adhered to the outlet of the nozzle for better flow control. The cell/matrix suspension is loaded into the internal channel of the 3-way device, which is kept refrigerated by means of an in line cooling system in order to avoid premature gelling of the Matrigel. A sodium alginate solution (Novamatrix Proonova SLG100, 0.25 g at 2% in distilled water) is injected into the external channel. To prevent the gelling of alginate in the microfluidic device due to the release of calcium by the cells in suspension, a calcium-free solution (Sorbitol 300 mM, Sigma-Aldrich) is used in the intermediate channel of the co-extrusion chip and serves as a barrier against calcium diffusion. The flow rates for the 3 solutions were of the order of 120 ml/h for the three channels (alginate solution, the sorbitol solution and the cell+matrix suspension). At these flow rates, the composite solution forms a liquid jet which fragments into droplets (approximately twice the size of the nozzle) due to the spontaneous Rayleigh-Plateau instability. To avoid subsequent coalescence of the train of droplets, an alginate filler part and a copper ring are connected to a high-voltage generator (2000 V). When the composite droplets come into contact with the calcium collecting bath (at 100 mM), the external layer of alginate gels. Therefore, the internal cell/matrix solution remains trapped inside a closed, spherical and permeable microcompartment. In the minutes that follow the encapsulation, the capsules are rinsed with medium (DMEM) to reduce the basal calcium concentration. Finally, they are transferred into a culture medium in suspension.

The passes of the experimental “Invention” arm correspond to re-encapsulations. These re-encapsulations are carried out by dissolving the alginate capsules using a short rinsing with ReleSR, followed by cellular dissociation using TrypLE (dissociation enzyme based on trypsin, ThermoFisher) for 20 minutes at 37° C. Next, the cells obtained were treated according to an encapsulation protocol according to the invention.

### Results:

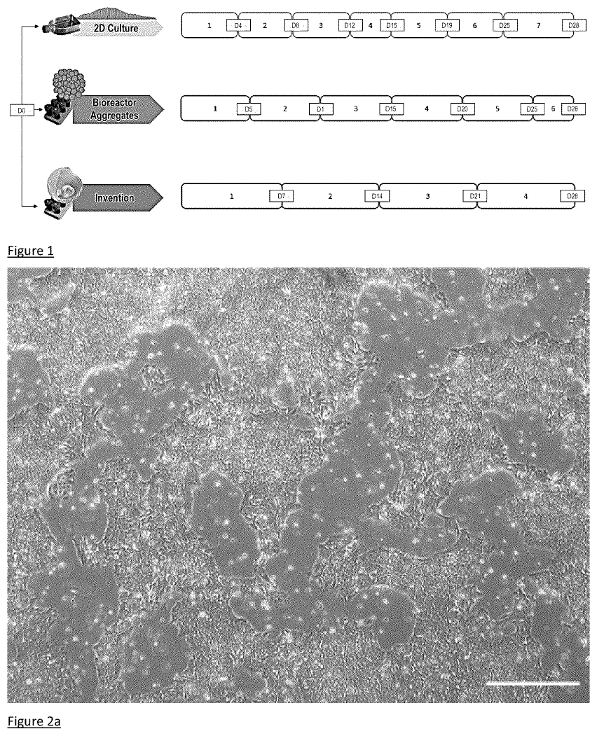

4 successive encapsulations, with a duration of 7 days each, were done. 6 successive passes were carried out for the “Bioreactor aggregates” arm and 7 successive passes were carried out for the “2D culture” arm. The sampling of cells on each pass and at 28 days allowed a comparative evaluation of the 3 culture arms over time (FIG. 1).

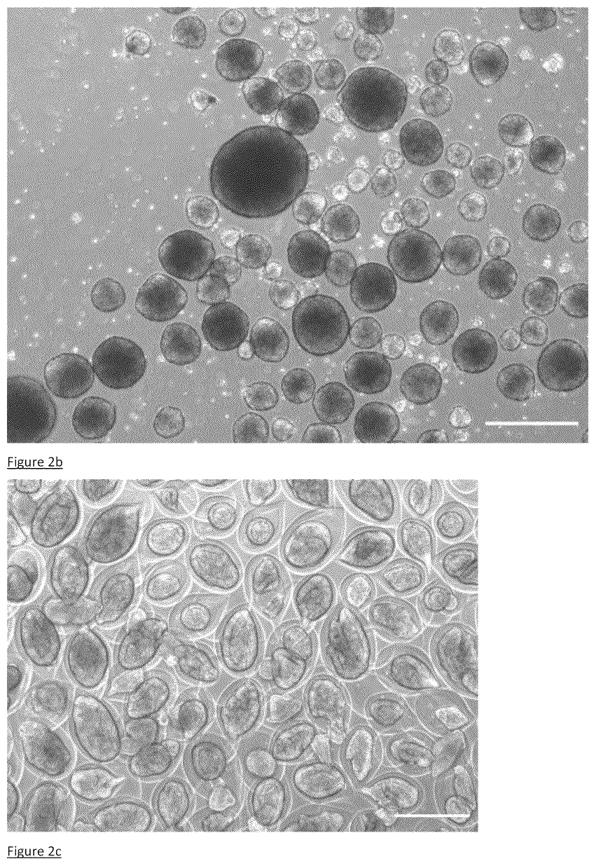

The evaluation by phase contrast microscopy confirms the successful formation of two-dimensional colonies, aggregates and encapsulated cysts of stem cells as was expected for the “2D culture” “Bioreactor aggregates” and “Invention” arms (FIG. 2a, 2b, 2c).

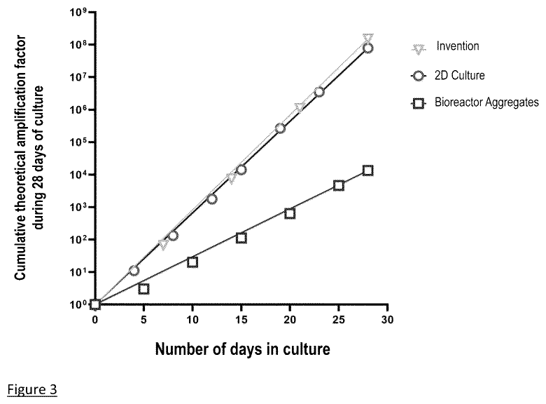

On each pass, the cells are counted using the cell counter (Nucleo Counter NC 3000), which makes it possible to establish the cell amplification factors during the culture (FIG. 3). The cumulative theoretical amplifications are 151 million, 71 million and 13,330 respectively for the experimental arms “Invention,” “2D culture” and “Bioreactor aggregates.” These cumulative amplification factors correspond to a number of average visible cell divisions in 28 days of 27.2; 26.2 and 13.7 respectively for the “Invention,” “2D culture” and “Bioreactor aggregates” culture arms. It is observed that the final cell amplification is higher in the experimental arm “Invention” compared to the 2 other experimental arms.

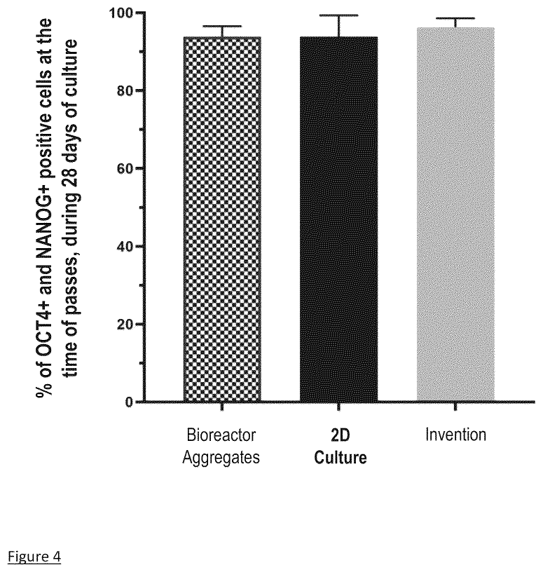

The 3 culture systems were successfully carried out according to the best standards, as suggested by the pluripotent markers OCT4 and NANOG similarly expressed in the 3 compared culture systems (FIG. 4).

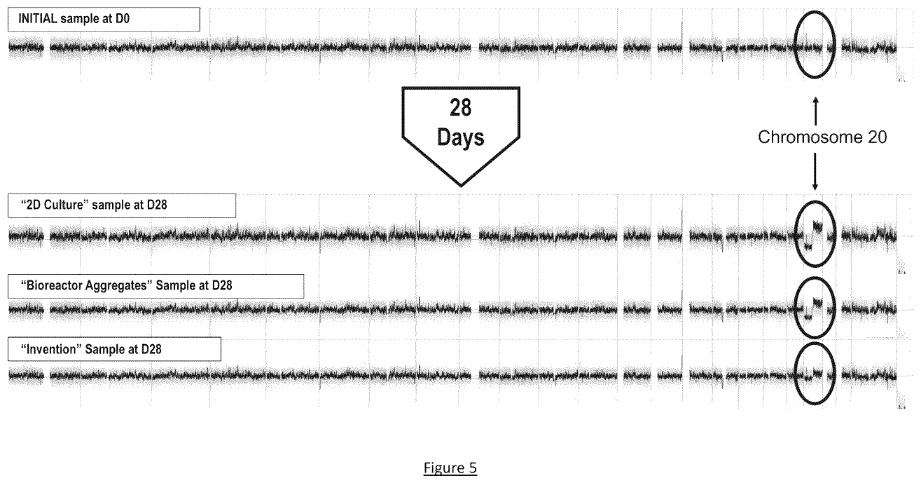

The genetic evaluation was first carried out by a very high definition SNP chip (CytoScan® HD Array Affymetrix, ThermoFisher) (FIG. 5). The appearance of a structural mutation is observed (chromosomal duplication encompassing the 20q11 area and a deletion) near 20 chromosome clearly visible for the final samples (D28) of the experimental arms “2D culture” (about 50% mutant cells) and “Bioreactor aggregates” (about 50% mutant cells). Similar profiles for refitting 20 chromosome for these 2 samples indicate that it does not involve independent events and that this mutation is inherited from a common ancestor. Thus, even if this mutation is undetectable during the initial sampling, this strongly suggests its presence at the initial moment of the experiment at a low percentage. For sample D28 of the “Invention” arm, a lower amplitude of the copy number is noted, which corresponds to a percentage of cells mutated in population less than 10%.

Digital PCR analyses were also carried out on each pass for all the experimental arms to detect the possible appearance of recurrent genetic mutations for the pluripotent stem cells (iCS-digital PSC 24 probes, StemGenomics). In particular, a PCR probe of this test made it possible to quantify over time the count of copies of the 20q11 chromosomal region (FIG. 6). The average count of copies of the 20q11 region increases over time in culture for the cells of the 3 experimental arms. This increase is greater and faster for the “2D culture” and “Bioreactor aggregates” arms compared to the “Invention” arm. Considering that the count of copies of the region 20q11 for each mutant cell is 3 (1 copy increase cf. FIG. 5), an average count of copies less than 2.2 corresponds to a percentage of mutant cells in the population of cells of less than 20%.

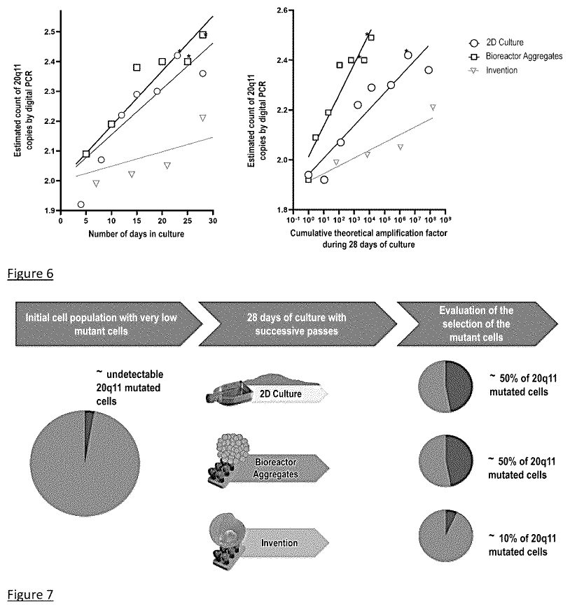

In total, the digital PCR and SNP chip results are consistent and suggest that the selection of mutant cells during the 28 days of culture was at least 5 times lower in the “Invention” arm relative to the “2D culture” and “Bioreactor aggregates” arms (FIG. 7). In particular, the encapsulated culture system (Invention) made it possible to produce, on average, 6.8 cell divisions per pass, while maintaining the percentage of mutant cells less than 20% for each encapsulation, or by putting the 4 encapsulations made end-to-end.

### Example 2

### Protocol:

In this example, two cell lines are used: a commercial line called iPSC-GHE (Gibco) and a transgenic line constitutively expressing the GFP fluorescent protein, called iPSC-AAVS1-GFP (Coriell, Allen Institute for Cell Science).

At the start of the experiment, the two lines are cultured independently in 2D. A karyotypic analysis by digital PCR (ICS-digital PSC 24 probes, Semenomics) reveals that the iPSC-GHE line has two karyotypic abnormalities with amplifications of the chromosomal regions 7q and 20q, whereas the iPSC-AAVS1-GFP line does not present any abnormality on the 24 zones studied (Fijack 8).

The use of the iPSC-GHE line having the amplifications of the 7q and 20q chromosomal regions is particularly relevant to test the positive selection over time of a mutant clone in a population of cells in culture. Indeed, the mutations by amplification of the 7q and 20q chromosomal region confers a growth advantage on the mutated clone; the greater the selection pressure of the culture system, the more the risk this clone will be rapidly selected and become predominant.

The encapsulated culture system in stirred suspension, “Invention,” was compared to the standard culture system in the field of pluripotent stem cell production: the culture in an unprotected stirred suspension in aggregate form, “Bioreactor aggregates,” over a period of 21 days.

The sample used to initiate in parallel the 2 experimental arms associated with the 2 culture systems, corresponds to a mixture of the iPSC-AAVS1-GFP and iPSC-GHE lines. The mixture corresponds to 80% of iPSC-AAVS1-GFP with 20% of iPSC-GHE.

On each pass, and for each experimental arm, cells are sampled to allow the performance of cytometric and genetic tests (See the results section). In particular, the analyses aim to monitor the development of the frequency of the iPSC-GHE population containing the karyotypic abnormalities within the culture.

The rate of the passes for each culture system scrupulously follows the optimal recommendations for each condition. Thus, the cultures in aggregates are passed every 5 days according to the supplier's recommendations (Minibio, ABLE® Bioreactor Systems) and the encapsulated cultures are passed every 7 days when the mean capsular confluence is between 50 and 100%.

All the cultures described below are carried out with the mTeSR1 plus culture medium (Stemcell Technologies). A 10 μM Rock inhibitor treatment is initiated during the first 24 hours after a pass.

All the cultures (Bioreactor aggregates and Invention) are held in a cell culture incubator at 37° C. and 5% CO₂.

The two experimental arms, “Bioreactor aggregates” and “Invention,” use 30 ml mini-bioreactors from the Minibio, ABLE® Bioreactor Systems brand; the stirring speed is constant at 55 rotations per minute for the “Bioreactor aggregates” condition and 100 rotations per minute for the “Invention” condition.

The 2 experimental arms, “Bioreactor aggregates” and “Invention,” use an enzymatic dissociation for successive passes: aggregates on the one hand and the encapsulated cysts on the other hand, are dissociated by use of a TryplE bath at 37° C. The cells and small groupings (clusters) of cells resulting from this dissociation are then used to inoculate a new culture.

For the encapsulations or re-encapsulations the cells are mixed with the Matrigel® before injection into the central microfluidic channel, the culture in aggregates does not require the use of extracellular matrix.

The “Bioreactor aggregates” culture is initiated with the same cell suspension used to inoculate the “Invention” culture but with an initial concentration of 175,000 cells per ml of medium, for a total of 10 ml of medium. The culture medium is completely changed on day 1 to remove the rock inhibitor.

Cultivation of the HiPSCs according to the invention (encapsulation according to the invention):

Before encapsulation, the 2D stem cell colonies of the iPSC-GHE and iPSC-AAVS1-GFP lines were dissociated using Accutase (StemCell Technologies). The two iPSCs lines were then mixed according to the previously indicated ratios (80%-20%) and this cell suspension itself was mixed in a 50/50 volume ratio with Matrigel at 4° C. to maintain the suspension in the liquid state.

The final concentration of cells in the cell/matrix solution was therefore between 0.4 and 1.0×10⁶ viable cells/ml, called encapsulation density. Ethylene-tetrafluoroethylene (ETFE) tubes are connected to the three inlets of a 3D printed microfluidic colaminar flow device. A microcapillary tip made of extruded and polished glass (with a nozzle diameter of about 100 μm for most experiments or a nozzle diameter of 150 μm) is adhered to the outlet of the nozzle for better flow control. The cell/matrix suspension is loaded into the internal channel of the 3-way device, which is kept refrigerated by means of an in line cooling system in order to avoid premature gelling of the Matrigel. A sodium alginate solution (Novamatrix Proonova SLG100, 0.25 g at 2% in distilled water) is injected into the external channel. To prevent the gelling of alginate in the microfluidic device due to the release of calcium by the cells in suspension, a calcium-free solution (Sorbitol 300 mM, Sigma-Aldrich) is used in the intermediate channel of the co-extrusion chip and serves as a barrier against calcium diffusion. The flow rates for the 3 solutions were of the order of 120 ml/h for the three channels (alginate solution, the sorbitol solution and the cell+matrix suspension). At these flow rates, the composite solution forms a liquid jet which fragments into droplets (approximately twice the size of the nozzle) due to the spontaneous Rayleigh-Plateau instability. To avoid subsequent coalescence of the train of droplets, an alginate filler part and a copper ring are connected to a high-voltage generator (2000 V). When the composite droplets come into contact with the calcium collecting bath (at 100 mM), the external layer of alginate gels. Therefore, the internal cell/matrix solution remains trapped inside a closed, spherical and permeable microcompartment. In the minutes that follow the encapsulation, the capsules are rinsed with medium (DMEM) to reduce the basal calcium concentration. Finally, they are transferred into a culture medium in suspension.

The passes of the experimental “Invention” arm correspond to re-encapsulations. These re-encapsulations are carried out by dissolving the alginate capsules using a short rinsing with ReleSR, followed by cellular dissociation using Accutase. Next, the resulting cells were treated according to an encapsulation protocol according to the invention.

### Results:

With a duration of 7 days each, 3 successive encapsulations were done and 4 successive passes were done for the “Bioreactor aggregates” arm. The sampling of cells on each pass and at 21 days allowed a comparative evaluation of the 2 culture arms over time (FIG. 9).

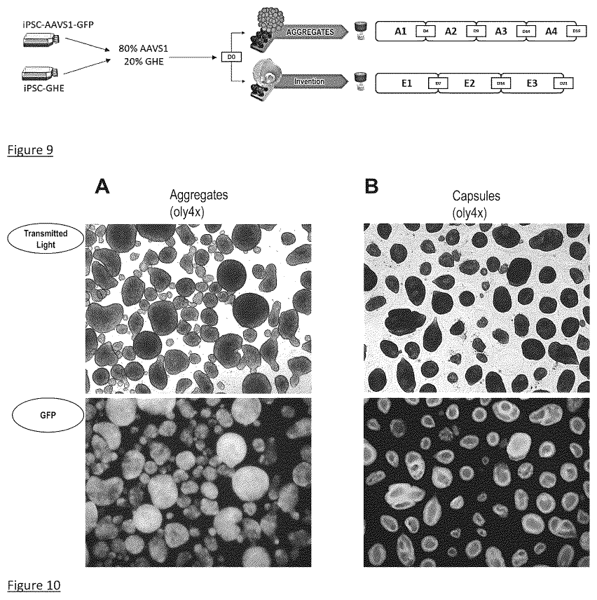

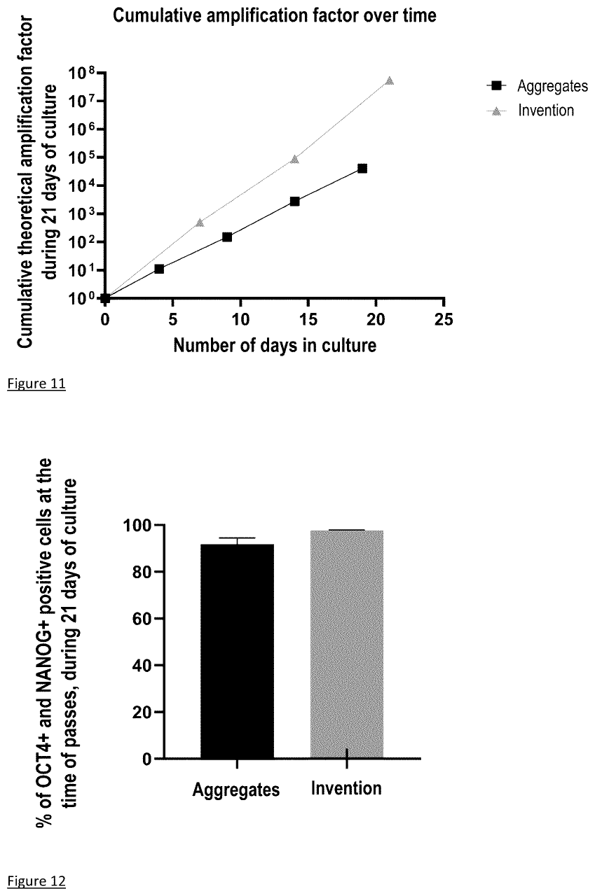

The evaluation by phase contrast microscopy confirms the successful formation of aggregates and encapsulated cysts of stem cells as was expected for the “Bioreactor aggregates” and “Invention” arms (FIG. 10).

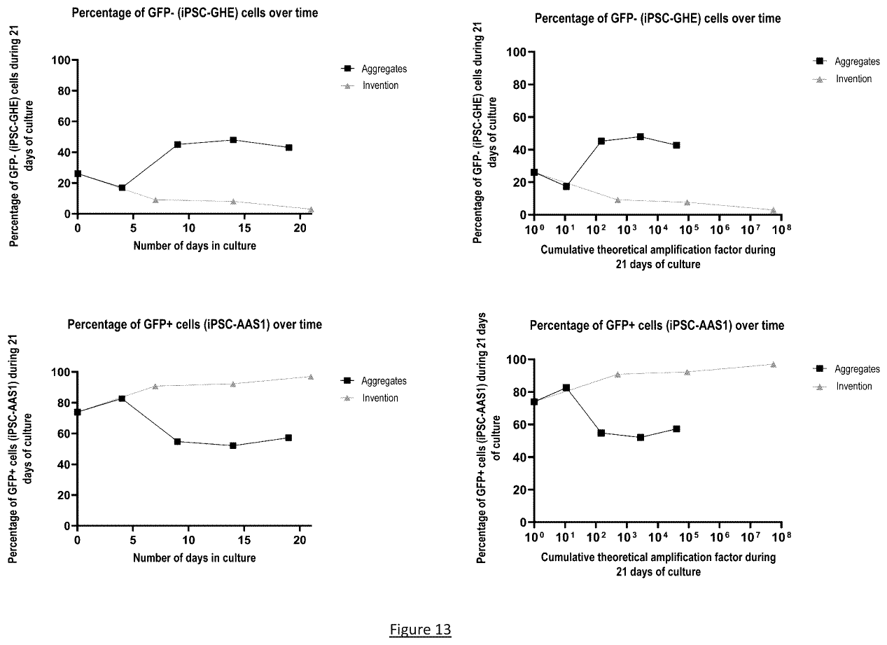

On each pass, the cells are counted using the cell counter (Nucleo Counter NC 3000), which makes it possible to establish the cell amplification factors during the culture (FIG. 11). The cumulative theoretical amplifications are 55,776,699 million and 40,481 respectively for the experimental arms “Invention” and “Bioreactor aggregates.” These cumulative amplification factors correspond to a number of average visible cell divisions in 21 days of 25.73 and 15.30 respectively for the “Invention” and “Bioreactor aggregates” culture arms. It is observed that the final cell amplification is higher in the experimental arm “Invention” compared to the “Bioreactor aggregates” experimental arms.

The 2 culture systems were successfully carried out according to the best standards, as suggested by the pluripotent markers OCT4 and NANOG similarly expressed in the 2 compared culture systems (FIG. 12).

A first flow-cytometry analysis was carried out to monitor the development of the population of the iPSC-GHE line within the cell culture. The iPSC-GHE (GFP negative) cells contain amplifications of the 7q and 20q chromosomal regions, which confer a selective advantage during cultivation of the hiPSC. The iPSC-AAVS1-GFP (GFP positive) cells do not contain a chromosomal anomaly.

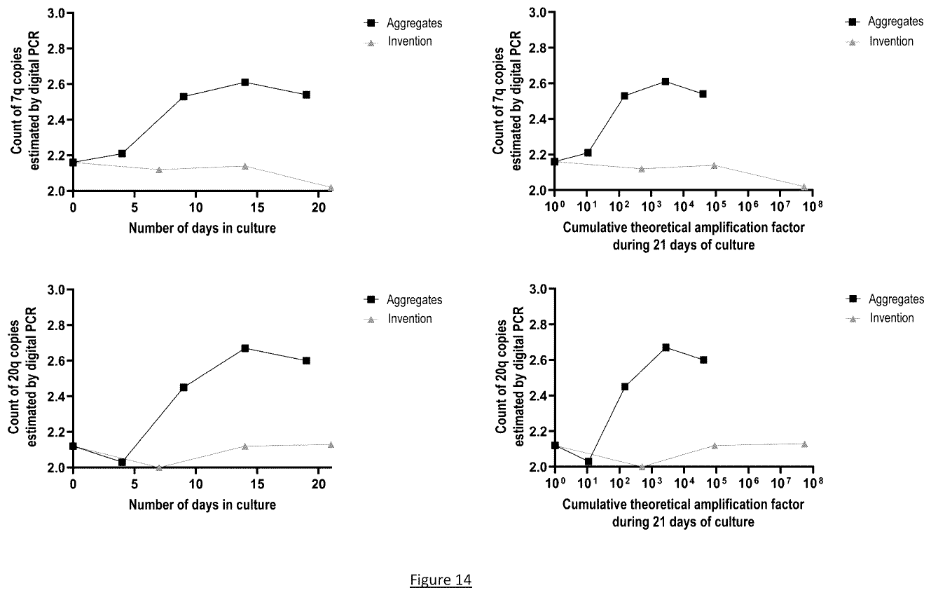

The flow-cytometry analysis on each pass for all experimental arms made it possible to quantify over time the frequency of iPSC-GHE and iPSC-AAVS1-GFP cells (FIG. 13) and therefore by extrapolation of the copy count of the chromosomal regions 7q and 20q. The frequency of the iPSC-GHE (negative GFP) population within the cell culture increases over time for the “Bioreactor aggregate” arm but decreases over time for the “invention” arm.

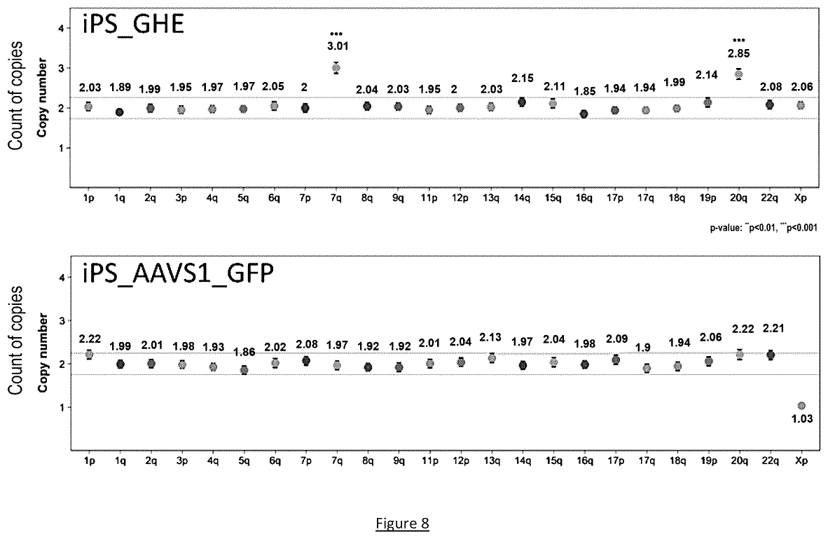

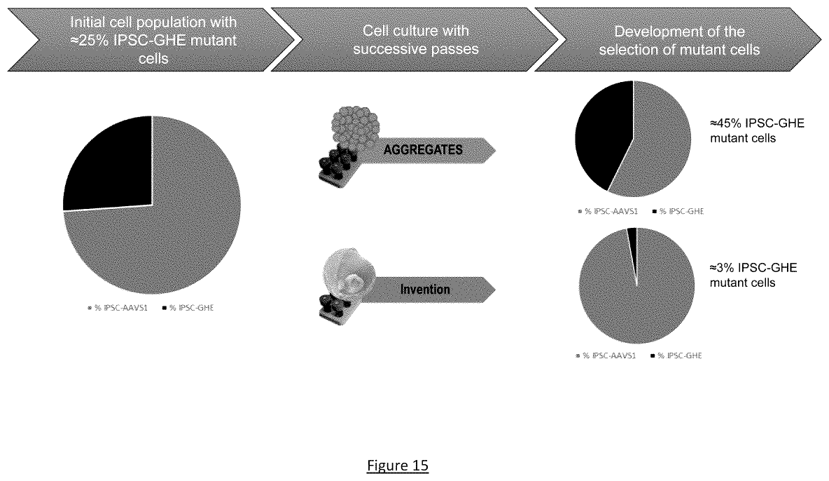

The flow cytometry analysis was then confirmed by a digital PCR analysis to detect the development of the genetic mutation rate in the population of pluripotent stem cell population (ICS-digital PSC 24 probes, StemGenomics). In particular, two PCR probes of this test made it possible to quantify over time the count of copies of the 7q and 20q chromosomal regions (FIG. 14). The average count of copies of the 7q and 20q regions increases over time in culture for the “Bioreactor aggregates” arm. For the “invention” arm, the average count of copies of the 7q region decreases over time, and the average count of copies of the 20q region decreases then increases very slightly over time but in a much less significant and less rapid way than for the “Bioreactor aggregates” arm. Considering that the count of copies of the 7q and 20q region for each iPSC-GHE mutant cell is 3 (1 copy increase cf. FIG. 8), an average count of copies equivalent to 2.3 corresponds to a percentage of mutant cells in the population of cells of 30%.

In total, the flow cytometry and digital PCR results are consistent and suggest that the selection of mutant cells during the 21 days of culture was at least 14.7 times lower in the “Invention” arm compared to the “Bioreactor aggregates” arm (FIG. 15). In particular, the encapsulated culture system (Invention) made it possible to produce, on average, 8.6 cell divisions per pass, while maintaining the percentage of mutant cells less than 20% (less than 3%) for each encapsulation, or by putting the 4 encapsulations made end-to-end.

1. A three-dimensional cellular microcompartment comprising at least one external hydrogel layer and inside said external layer at least one layer of cells and/or at least one cellular base layer, characterised in that less than 20% of the total population of cells present in the microcompartment are cells having at least one mutation.

2. The three-dimensional cellular microcompartment according to claim 1, characterised in that the cells represent more than 50% by volume relative to the volume of the microcompartment, preferably more than 70% by volume relative to the volume of the microcompartment.

3. The three-dimensional cellular microcompartment of claim 1, characterised in that the mutation(s) are chosen from genetic mutations and epigenetic mutations.

4. The three-dimensional cellular microcompartment of claim 1, characterised in that the mutation(s) are functional mutations.

5. The three-dimensional cellular microcompartment of claim 1, characterised in that at least one mutation is an oncogenic mutation.

6. The three-dimensional cellular microcompartment of claim 1, characterised in that less than 20% of the cells are cells having at least one mutation of the P53 gene and/or at least one mutation by amplification of the 20q chromosomal region and/or at least one mutation by amplification of the 7q chromosomal region.

7. The three-dimensional cellular microcompartment of claim 1, characterised in that less than 20% of the cells are cells having at least one mutation by amplification of the 20q11 chromosomal region.

8. The three-dimensional cellular microcompartment of claim 1, characterised in that the cells having at least one mutation represent between 0 and 10% of the total population of cells present in the microcompartment, preferably between 0 and 5%.

9. The three-dimensional cellular microcompartment of claim 1, characterised in that the cells are organised in the form of a tissue or a micro-tissue.

10. The three-dimensional cellular microcompartment of claim 1, characterised in that it comprises an internal lumen.

11. The three-dimensional cellular microcompartment of claim 1, characterised in that it also comprises, between (a) the cell layer(s) and/or the cellular base layer(s) and (b) the hydrogel layer, at least one intermediate layer of isotonic aqueous solution and/or comprising extracellular matrix elements.

12. The three-dimensional cellular microcompartment of claim 1, characterised in that the intermediate isotonic aqueous solution layer is an extracellular matrix layer.

13. The three-dimensional cellular microcompartment of claim 11, characterised in that the intermediate layer of isotonic aqueous solution has a Young's modulus between 0.05 and 3 kDa.

14. The three-dimensional cellular microcompartment of claim 1, characterised in that the cells are human or animal cells.

15. The three-dimensional cellular microcompartment of claim 1, characterised in that it comprises successively organised around a lumen:
at least one layer of cells and/or at least one cellular base layer;
an intermediate layer of isotonic aqueous solution; and
an external hydrogel layer.

16. The three-dimensional cellular microcompartment of claim 1, characterised in that the cells are human or animal induced pluripotent stem cells (iPSC), and/or human or animal multipotent cells, and/or human or animal progenitor cells and/or human or animal differentiated cells.

17. The three-dimensional cellular microcompartment of claim 1, characterised in that it is closed.

18. The three-dimensional cellular microcompartment of claim 1 characterised in that the external layer comprises alginate.

19. The three-dimensional cellular microcompartment of claim 1, characterised in that it has the shape of an ovoid, a cylinder, a spheroid or a sphere.

20. The three-dimensional cellular microcompartment of claim 1, characterised in that it comprises at least 20 cells, preferentially at least 1000 cells.

21. The three-dimensional cellular microcompartment of claim 1, characterised in that the cells present in the microcompartment were obtained after at least two cell division cycles after encapsulation in an external hydrogel layer of at least one cell, preferentially between 1 and 50 cells.

22. The three-dimensional cellular microcompartment of claim 1, characterised in that the cells present in the microcompartment were obtained after at least 5 cell division cycles after the encapsulation in an external hydrogel layer of at least one cell, preferentially between one and fifty cells.

23. An assembly of at least two three-dimensional cellular microcompartments, each microcompartment comprising at least one external hydrogel layer and inside said external layer at least one layer of cells and/or at least one cellular base layer, characterised in that less than 20% of the cells constituting the total population of cells present in all the microcompartments are cells having at least one mutation.

24. The assembly of microcompartments of claim 23, characterised in that at least one microcompartment is the three-dimensional cellular microcompartment of claim 1.

25. The assembly of microcompartments of claim 24, characterised in that the microcompartments are arranged in a culture medium in a closed bioreactor.

26. A method for preparing the three-dimensional cellular microcompartment of claim 1 or an assembly of the three-dimensional cellular microcompartments, the method comprising the following steps:
(a) preparing a suspension of cells comprising single cells and/or at least one cluster of cells in an isotonic medium, preferentially a culture medium containing an apoptosis inhibitor;
(b) encapsulating the cellular suspension in a hydrogel layer;
(c) preferentially culturing the resulting microcompartments in an isotonic solution containing an apoptosis inhibitor;
(d) preferentially rinsing the microcompartments, so as to remove the apoptosis inhibitor;
(e) culturing the microcompartments in an isotonic solution for at least two cell division cycles, and
(f) optionally recovering the resulting cellular microcompartments;
said method being characterised in that all of the cells initially encapsulated in step (b) represent a volume less than 50% of the volume of the microcompartment in which they are encapsulated.

27. The method of claim 26, characterised in that each cell cluster initially encapsulated in step (b) has a larger dimension less than 20% of the largest dimension of a microcompartment in which it is encapsulated.

28. The method of claim 26, characterised in that it comprises a step of mixing the cells with an extracellular matrix, either between step (a) and step (b), or simultaneously with the encapsulation in step (b).

29. The method of claim 26, characterised in that steps (c), (d) and (e) are carried out under continuous or sequential stirring.

30. The method of claim 26, characterised in that it is implemented in a closed bioreactor.

31. The method of claim 26, characterised in that the method comprises at least one re-encapsulation of the cells after step (e).

32. The method of claim 31, characterised in that the method comprises between 2 and 15 re-encapsulations of the cells.

33. The method of claim 31, characterised in that each re-encapsulation corresponds to a pass.

34. The method of claim 31, characterised in that the re-encapsulation comprises the following steps:
(i) removing the external hydrogel layer,
(ii) resuspending the cells that were contained in the microcompartment so as to obtain single cells and/or at least one cluster of cells in an isotonic medium, preferentially a culture medium containing an apoptosis inhibitor,
(iii) encapsulating the cellular suspension in a hydrogel layer;
(iv) preferentially culturing the resulting microcompartments in an isotonic solution containing an apoptosis inhibitor;
(v) preferentially rinsing the microcompartments, so as to remove the apoptosis inhibitor;
(vi) culturing the microcompartments in an isotonic solution for at least one cell division cycle; and
(vii) optionally recovering the resulting cellular microcompartments.

35. The method of claim 26, characterised in that for each microcompartment, the single cells represent less than 50% by number of all of the cells initially encapsulated in step (b).

36. The method of claim 26, characterised in that prior or simultaneously to step (a), the method comprises a step of dissociation of the cells by chemical, enzymatic or mechanical dissociation.

37. The method of claim 26, characterised in that it comprises one or more steps of removing the microcompartments comprising mutant cells.

38. The method of claim 26 wherein the method maintains the genomic integrity of cells during their amplification.

39. The three-dimensional cellular microcompartment of claim 1 useful for maintaining genomic integrity of cells during their amplification.
